(19) United States
(12) Patent Application Publication
(10) Pub. No.: US 2008/0312510 A1
(43) Pub. Date: Dec. 18, 2008

(54) WELLNESS PROGRAMS, INCLUDING COMPUTER IMPLEMENTED WELLNESS PROGRAMS

(76) Inventor: S. Michael Ross, Merion Station, PA (US)

Correspondence Address: PERKINS COIE LLP;PATENT-SEA, P.O. BOX 1247, SEATTLE, WA, 98111-1247, US

(21) Appl. No.: 11/763,329

(22) Filed: Jun. 14, 2007

(51) Int. Cl. A61B 5/00

(52) U.S. Cl. 600/300

(57) ABSTRACT

Aspects of the wellness programs are disclosed. For example, one aspect of the invention is directed toward a computing system that includes a health question generation component configured to provide a set of health questions to each individual in a group. The system further includes a health input tracking component configured to track health inputs received from each individual and to prompt individuals to provide input for one or more health questions to which selected individual(s) have not responded. The system still further includes a health reporting component configured to report information related to health profiles. In selected embodiments, the system can include a readiness question generation component configured to provide a set of readiness questions to one or more individuals, wherein the set of readiness questions are formatted to assess a readiness of the one or more individuals to take a selected action.

### TECHNICAL FIELD

The present invention relates to wellness programs, including associated computer methods and systems.

### BACKGROUND

Healthcare cost can be expensive. One way to reduce healthcare costs is through healthcare screening where health related information is collected from individuals and used to improve healthcare management. For example, screening can be used to provide a health risk factor assessment or to provide early detection of disease. This information can allow an individual to get appropriate care in a timely manner. In some cases, receiving early treatment can reduce overall healthcare cost as compared to those costs when certain diseases go undetected or selected risk factors are ignored. Additionally, in some cases timely treatment can provide a better quality of health and a better quality of life for the individual. In turn, this can result in an individual being more productive on the job, reduce absenteeism, etc.

Unfortunately, healthcare screening can be difficult to conduct, expensive, and time consuming. For example, in some cases healthcare screening requires trained personnel to contact each individual and conduct the screening. Additionally, it can be difficult to get an individual to commit to the block of time required for the person to person screening session. In some cases, this can cause individuals to simply avoid the screening process.

In other cases, the individual can be asked to complete a questionnaire that solicits information about health related issues. However, in some cases, once an individual completes a questionnaire trained personnel are required to review the completed questionnaire to make a health assessment. Additionally, these questionnaires can be lengthy and time consuming for the individual to complete. Consequently, it can be easy for the individual to delay the completion of the questionnaire, forget to take/finish the questionnaire, or avoid taking the questionnaire altogether. Accordingly, in some cases the screening process can be expensive and it can be difficult to get individuals to complete the screening process.

### BRIEF DESCRIPTION OF THE DRAWINGS

FIG. 1 is a partially schematic block diagram that illustrates a computing environment suitable for implementing a wellness program in accordance with embodiments of the invention.

FIG. 2 is a partially schematic illustration of selected computing system components suitable for implementing a wellness program in accordance with certain embodiments of the invention.

FIG. 3 is a flow diagram that illustrates a portion of a method for administering a wellness program in accordance with selected embodiments of the invention.

FIG. 4 is a partially schematic timeline of at least a portion of a wellness program in accordance with an embodiment of the invention.

FIG. 5 is a partially schematic illustration of an email display in accordance with certain embodiments of the invention.

FIG. 6 is a partially schematic illustration of a display of a network site that provides questions in accordance with selected embodiments of the invention.

FIG. 6A is a partially schematic illustration of a display of a network site that provides an indication that not all of the questions shown in FIG. 6 have been answered in accordance with certain embodiments of the invention.

FIG. 7 is a partially schematic illustration of an email display reporting information in accordance with selected embodiments of the invention.

### DETAILED DESCRIPTION

In the following description, numerous specific details are provided in order to give a thorough understanding of embodiments of the invention. One skilled in the relevant art will recognize, however, that the invention may be practiced without one or more of the specific details, or with other methods, components, materials, etc. In other instances, well known structures, materials, or operations are not shown or described in order to avoid obscuring aspects of the invention.

References throughout the specification to “one embodiment” or “an embodiment” means that a particular feature, structure, or characteristic described in connection with the embodiment is included in at least one embodiment of the present invention. Thus, the appearances of the phrase “in one embodiment” or “in an embodiment” in various places throughout the specification are not necessarily all referring to the same embodiment. Furthermore, the particular features, structures, or characteristics may be combined in any suitable manner in one or more embodiments.

Aspects of the present invention are directed generally toward wellness programs. For example, one aspect of the invention is directed toward a computing system for administering at least a portion of a wellness program that includes a contact information receiving component configured to receive contact information for a group of individuals. The system further includes a health question generation component configured to contact each individual in the group of individuals and to provide a set of health questions to each individual. The set of health questions is formatted to determine a health profile of each individual that responds to the set of health questions. The system still further includes a health input receiving component configured to receive health inputs from the individuals in response to the set of health questions. The system yet further includes a health input tracking component configured to track health inputs received from each individual and to determine one or more health questions in the set of health questions to which a selected individual has not responded. The tracking component is further configured to prompt the selected individual to provide input for the one or more health questions to which the selected individual has not responded. The system still further includes a health analysis component configured to analyze the health inputs to determine a health profile for each individual in a portion of individuals that respond to the set of health questions. The system yet further includes a health reporting component configured to report information related to the health profile of the individuals in the portion of individuals that respond to at least a portion of the set of health questions.

In other embodiments, the system can still further include a readiness question generation component configured to determine one or more individuals in the portion of individuals to provide a set of readiness questions based at least in part on a portion of the health profile of the one or more individuals. The set of readiness questions are formatted to assess a readiness of the one or more individuals to take a selected action. The readiness question generation component can also be configured to provide the set of readiness questions to the one or more individuals. The system can yet further include a readiness input receiving component configure to receive readiness input from the one or more individuals in response to the set of readiness questions. The system can still further include a readiness input tracking component configured to track readiness inputs received from the one or more individuals and to determine one or more readiness questions in the set of readiness questions to which a selected individual has not responded. The tracking component can be further configured to prompt the selected individual to provide input for the one or more readiness questions to which the selected individual has not responded. The system can yet further include a readiness analysis component configured to analyze the readiness input to determine the readiness of at least one individual to take the selected action based on the readiness input received and a readiness reporting component configured to report information related to the readiness of the at least one individual to take the selected action.

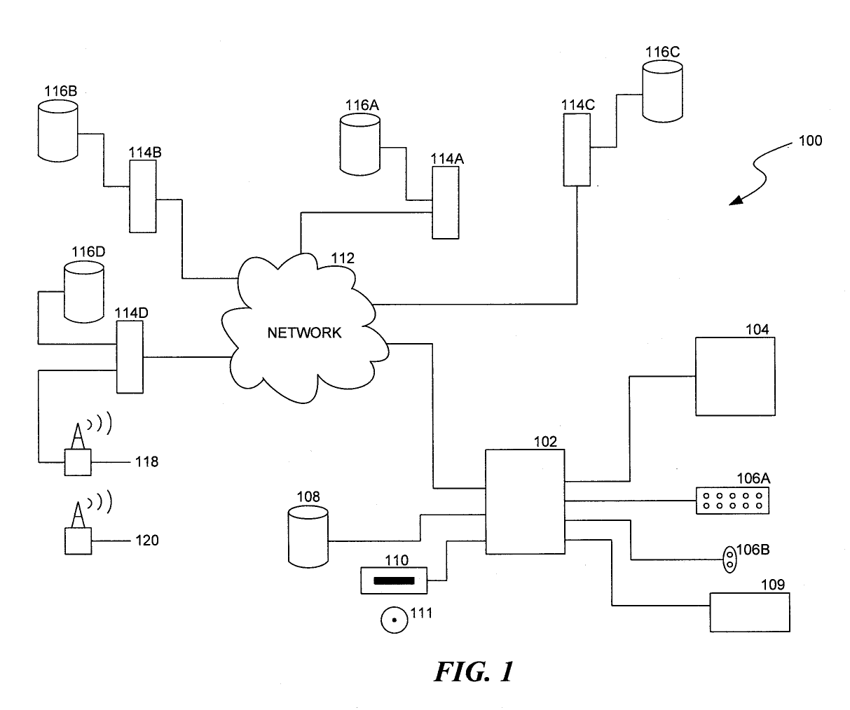

FIG. 1 is a block diagram that illustrates a computing environment or computing system 100 suitable for implementing a process related to one or more wellness programs in accordance with embodiments of the invention. The computing system 100 can include a computer 102 that can be operably connected or coupled to a display 104 and one or more input devices, for example, a keyboard 106a and a pointing device 106b (e.g., a mouse). Additionally, the computer 102 can communicate with one or more storage devices (e.g., a hard drive 108 with one or more databases) and one or more devices 110 for reading other types of computer readable mediums (e.g., devices for reading disks 111). The computer 102 can also communicate directly with other devices 109, for example, a phone and/or fax system, or with other devices or systems via a network 112 (e.g., via the Internet). For example, in the illustrated embodiment the computer 102 can communicate with other computers 114a-d and/or other databases 116a-d via the network 112. Additionally, in selected embodiments the computing system 100 can include one or more wireless devices 120 (e.g., a wireless computing device such as a personal data assistant) and one or more wireless transmitter/receiver 118 (e.g., a service provider connected to the internet) configured to communicate with one another. In other embodiments, the computing environment can have other arrangements, including more, fewer, and/or different components.

For example, the computing system on which the system is implemented may include a central processing unit, memory, input devices (e.g., keyboards, keypads, and/or pointing devices), output devices (e.g., display devices, printers, etc.), and storage devices (e.g., disk drives). The memory and storage devices are computer-readable media that may contain instructions that implement the system. In addition, the data structures and message structures may be stored or transmitted via a data transmission medium, such as a signal on a communication link. Various communication links may be used, such as the Internet, a local area network, a wide area network, a point-to-point dial-up connection, a cell phone network, and so on.

Embodiments of the system may be implemented in various operating environments that include personal computers, server computers, hand-held or laptop devices, multiprocessor systems, microprocessor-based systems, programmable consumer electronics, digital cameras, network PCs, minicomputers, mainframe computers, distributed computing environments that include any of the above systems or devices, and so on. The computer systems may be cell phones, personal digital assistants, smart phones, personal computers, programmable consumer electronics, digital cameras, and so on.

The system may be described in the general context of computer-executable instructions, such as program modules, executed by one or more computers or other devices. Generally, program modules include routines, programs, objects, components, data structures, and so on that perform particular tasks or implement particular abstract data types. Typically, the functionality of the program modules may be combined or distributed as desired in various embodiments.

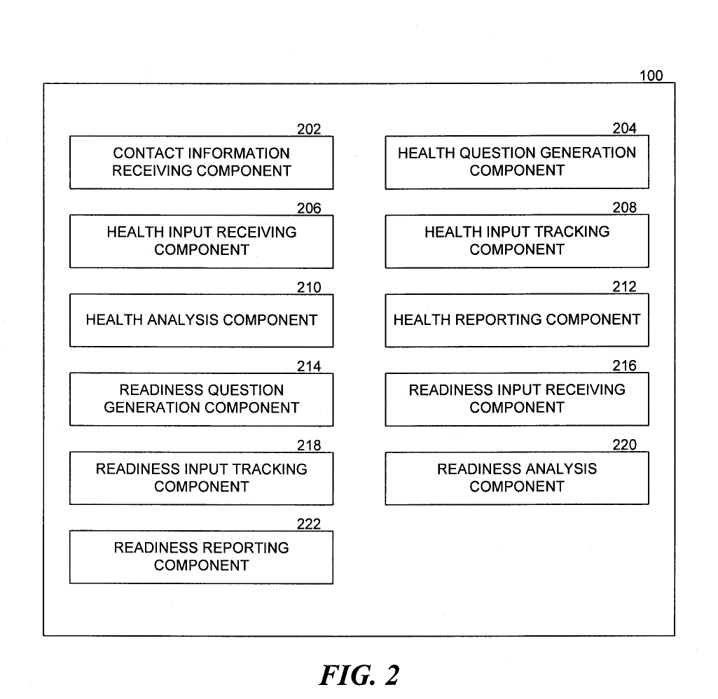

FIG. 2 is a partially schematic illustration of selected components in a portion of a computing system 100 suitable for implementing a wellness program in accordance with certain embodiments of the invention. In FIG. 2, the portion of the computing system 100 includes a contact information receiving component 202, a health question generating component 204, a health input receiving component 206, a health input tracking component 208, a health analysis component 210, and a health reporting component 212. The computing system 100, shown in FIG. 2, also includes a readiness question generation component 214, a readiness input receiving component 216, a readiness input tracking component 218, a readiness analysis component 220, and a readiness reporting component 222.

In selected embodiments, one or more of the computing system components shown in FIG. 2 can be used to administer at least a portion of a wellness program that collects health information from a group of individuals. In other embodiments, one or more of the computing system components can be used to administer at least a portion of a wellness program that collects readiness information from a group of individuals. For example, the readiness information can include information associated with the readiness of one or more individuals to take an action (e.g., the readiness to change a behavior, seek assistance, etc.). In some embodiments, the computing system components can be used to administer at least a portion of a wellness program that analyzes the collected information and/or reports related data.

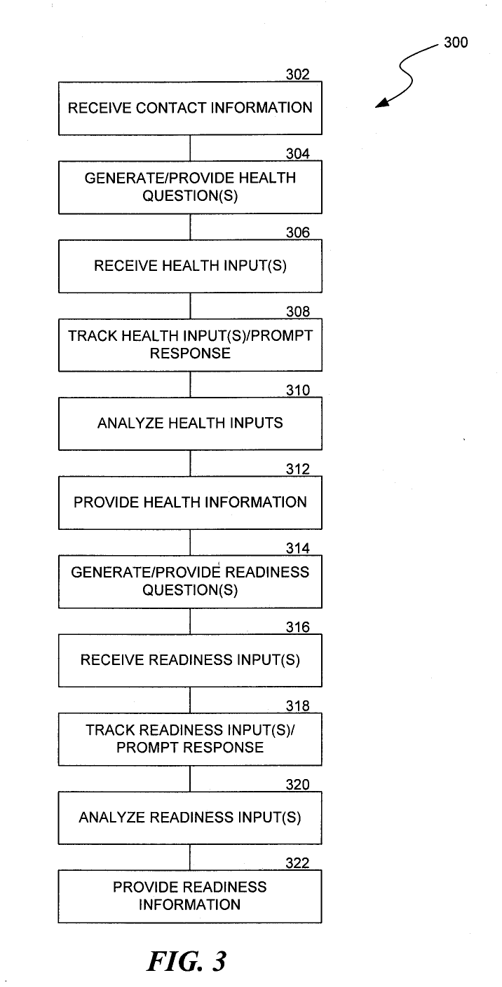

FIG. 3 is a flow diagram that illustrates a portion of a method 300 for administering a wellness program in accordance with selected embodiments of the invention. For example, in certain embodiments the contact information receiving component 202 (shown in FIG. 2) can receive contact information (process portion 302) for one or more individuals. For instance, in selected embodiments the contact information can include contact information for a group of individuals at a business entity. The contact information can include various types of information, such as work telephone number, home telephone number, work address, home address, work email, home email, electronic employee information website or electronic bulletin board information, text messaging address, and/or the like.

Additionally, in selected embodiments other information related to each individual can be included with the contact information. For example, in selected embodiments where the individuals have given consent, other information can be included along with the contact information such as known health information associated with the individuals and/or insurance information. The contact information and other information can be stored in one or more databases associated with the contact information receiving component and/or other components of the computing system.

The health question generation component 204 can use the contact information to generate and/or provide a set of health questions to at least a portion of the individuals (e.g., a group of individuals) for which contact information is received (process portion 304). For example, health questions can include various questions that are formatted, formulated, or designed to solicit information that can be used to determine a health profile for each individual that responds to the set of health questions. For example, the health profile can include an assessment of an individual's health, an individual's behavior/lifestyle attributes associated with health, and/or an individual's various health risk factors (e.g., risk factors based on genetics or family history, medical history, and lifestyle/behavior).

In selected embodiments, the health question generation component 204 can include a single stored question set that is provided to each individual in a group or portion of individuals for which contact information has been received (e.g., questions stored in a database). In other embodiments, the health question generation component 204 can include multiple stored question sets, and the health question generation component 204 can provide one or more selected question sets to each individual based on the contact information and/or other information provided by the individual (e.g., known information provided with the contact information and/or based on inputs received in response to selected questions provided to the individual). In still other embodiments, the health question generation component 204 can include multiple stored questions, and provide each individual with a selected question set generated from the stored questions based on the contact information and/or other information provided by the individual.

For example, in a selected embodiment an individual can be asked “do you smoke?” If the individual answers yes, the health question generation component 204 can then ask the individual how many times a day do they smoke, how long has he or she smoked, etc. If the individual answers no, the health question generation component 204 can simply move on to another question (e.g., do you drink alcohol?).

For example, in certain embodiments the health question generation component 204 can contact individuals in the group of individuals for which contact information was received and provide basic information regarding the wellness program, including the purpose of the program, how the program will be administered, benefits associated with the program, privacy information associated with the program, information about health questions, information about readiness questions which are discussed in further detail below, and/or incentives for answering health or readiness questions (e.g., a cash reward). In selected embodiments, the health question generation component 204 can contact individuals via an automated telephone call (e.g., an automated phone call that provides a recorded message) or other computer media, such as a computer generated display (e.g., information formatted to be provided by a computer display device). In certain embodiments, a computer generated display can include an e-mail, a text message, an electronic bulletin board, a computer information page, and/or a network website where information is posted for a selected group of people. In some embodiments, computer media can include various audio input/output devices (e.g., speakers, microphones, voice recognition software, etc.).

In selected embodiments, the health question generation component 204 and/or other components of the computing system can include various identification and verification processes throughout some or all portions of the method 300 for administering a wellness program. For example, in selected embodiments an individual can be required to provide a password or employee ID before sensitive information is provided/collected via a telephone call and/or other computer media (e.g., including processes that provide HIPPA compliance). For example, in selected embodiments where an automated telephone call is used to contact the individual, a discrete callback message can be left (e.g., on an answering machine or with a person answering the phone) unless proper identification/verification information is provided. U.S. patent application Ser. No. 11/593,945, entitled HEALTHCARE MANAGEMENT SYSTEMS AND ASSOCIATED METHODS, filed Nov. 6, 2006, which is fully incorporated herein by reference, includes some identification/verification processes (as well as other automation processes) suitable for use with selected embodiments of the method 300 for administering a wellness program.

During the initial contact and/or during a subsequent contact the health question generation component 204 can provide additional information about the wellness program and/or provide a set of health questions. For example, in certain embodiments the health question generation component 204 can provide health questions to individuals via an automated telephone call or other computer media message that directs individual(s) to a website that provides the individual with the appropriate set of health questions once the individual provides selected identifying information (e.g., which can serve to verify the individual's identity). In still other embodiments, the health question generation component 204 can use an automated phone process or other computer media to provide a set of one or more health questions to an individual by providing a “call in” telephone number that presents health questions to the individual via an automated phone process. In other embodiments, the automated telephone call or other computer media can provide the individual with the capability of scheduling a time to receive an automated phone call during which the individual can respond to a question set provided via the telephone system.

For example, in selected embodiments a computer media message can provide a hyperlink to a website that allows the individual to schedule a time to receive an automated phone call during which the individual can respond to health questions. In other embodiments, an automated telephone call can provide an individual with the ability to select (e.g., via touchtone keypad input(s) and/or voice recognition software) a time to receive a second automated phone call during which the individual can respond to health questions. In still other embodiments, the health question generation component 204 can simply contact the individual via an automated telephone process, allow the individual to verify their identity (e.g., via an employee ID or password), and provide the individual with the set of questions in a format that allows the individual to respond via the touchtone keypad on the phone and/or via voice recognition software.

In selected embodiments, the health input receiving component 206 can be configured to receive and/or store health input(s) (process portion 306), for example, from individuals in response to the set of health questions. For example, in certain embodiments one or more the individuals in the group of individuals can be provided health questions via an automated phone call (e.g., initiated by the computing system or via a “call in” number provided to the individual(s)) and the individual(s) can respond to at least a portion of the health questions (e.g., provide health inputs) via an automated telephone process (e.g., via touchtone keypad inputs and/or voice recognition software) which can be received and/or stored by the health input receiving component 206 (e.g., in a database).

In other embodiments, the health questions can be provided to one or more individuals via a computer generated display presented on a computer display device. The individual(s) can respond to the questions (e.g., provide health inputs) using a computer input device. For example, the individual can respond to the questions presented on a computer generated display (e.g., a webpage) or via speaker(s) using one or more computer input devices such as a mouse, keyboard, keypad, and/or audio devices (e.g., using microphone(s) and voice recognition software). In selected embodiments, the health input receiving component 206 can receive and/or store these inputs.

In certain embodiments, a health input tracking component 208 can be configured to track health inputs (process portion 308) received from each individual. For example, the health input tracking component 208 can be configured to determine one or more questions in the set of health questions to which a selected individual has not responded. In selected cases, this can include determining that an individual has not answered any health questions, has not scheduled a time to receive an automated call, has not accessed a “call in” phone number, has not visited a network site, and/or the like. In selected embodiments, the health input tracking component 208 can be further configured to prompt the selected individual to respond to, or provide input for, the one or more questions to which the selected individual has not responded (process portion 308).

For example, the health input tracking component 208 can be configured to prompt a selected individual to answer any unanswered questions before the selected individual exits/completes the set or series of health questions or health survey. For instance, the selected individual can select “submit” (e.g., via a phone input or via an input on a website) when the individual reaches the end of the survey. The health input tracking component 208 can prompt the individual to answer any unanswered questions by highlighting, marking, and/or otherwise indicating the questions which remain unanswered, and/or presenting a new list of questions that includes only the unanswered questions (e.g., via the website or phone).

In other embodiments, once a selected individual has exited the survey without answering all of the questions, the health input tracking component 208 can be configured to prompt the selected individual to return to the medium in which the questions were originally presented (e.g., the same website and/or the same phone contact used to conduct the initial portion of the survey) to respond to any unanswered questions. For example, in certain embodiments the health input tracking component 208 can send an email or place an automated call reminding the selected individual to return to the medium and complete the survey and/or provide the individual with the option of completing the survey at the time the reminder is received (e.g., via an automated phone process and/or a hyperlink to a website/network site). In selected embodiments, this feature can allow an individual to complete the survey in parts (e.g., answering a portion of the questions during a first time period and answering a second portion of the questions during one or more subsequent time periods).

In still other embodiments, the input tracking component 208 can prompt an individual to complete the survey, but allow the individual to use a different medium than originally used. For example, in selected embodiments a selected individual can complete a portion of the survey via a website during a first period of time. Subsequently (e.g., a few days later), the input tracking component 208 can then initiate an automated phone call prompting the individual to complete the survey via phone (e.g., by providing the unanswered questions via phone and allowing the individual to respond via a touch tone keypad or voice recognition).

In selected embodiments, the input tracking component 208 can prompt the individual to answer unanswered questions, a portion of the health question generation component 204 can provide the questions, and a portion of the health input receiving component 206 can receive inputs provided in response to the unanswered questions. In certain embodiments, the input tracking component 208 can provide multiple prompts to an individual over a selected period of time, for example, when the individual does not answer all of the unanswered questions after each prompt and/or does not respond to any of the prompts. In still other embodiments, after an individual fails to complete the survey within a selected period of time and/or after a selected number of prompts, the input tracking component 208 can provide notice to an operator or attendant who can follow up with the individual (e.g., via a person to person phone call).

In certain embodiments, a health analysis component 210 can be configured to analyzing health inputs (process portion 310) to determine a health profile for each individual in a portion of individuals that respond to the set of health questions. The health profile can include health information related to an individual's current health and/or risk factors for developing medically significant conditions in the future (e.g., risk for diabetes, cancer, etc.). For example, in certain embodiments the set of health questions can include question(s) related to whether anyone in an individual's family has had diabetes, whether an individual is overweight or has had a weight problem, whether an individual has been told that he/she has had problems with lipid levels, whether an individual has experienced a recent change in fluid consumption or thirst, whether an individual has experienced a recent change in urination frequency, how many times a week an individual exercises and for what duration, and/or the like. If the inputs received in response to these questions indicate that an individual has a family history of diabetes, is overweight, has had problems with lipid levels, reports an increased thirst level, reports frequent urination, and reports that he/she does not exercise, the health analysis component 210 might determine that the individual is at risk for, or has a selected risk level (e.g., medium or high risk level) of developing diabetes.

A health reporting component 212 can be configured to report or provide information related to the health profile of the individuals in the portion of individuals that respond to at least a portion of the set of health questions (process portion 312). For example, in selected embodiments information related to the health profile of an individual can be reported to the individual via an automated phone call (e.g., after an appropriate identification and verification process) or via other computer media, such as email, a password protected webpage, and/or the like. In other embodiments, the health reporting component 212 can provide information to an individual by providing a “call in” number or scheduling a call back time that will allow an individual to receive the health information via an automated phone process. In still other embodiments, the health reporting component 212 can automatically print mailers to the individuals providing the related information.

In yet other embodiments, the health reporting component 212 can report the related information to a person or provider who can call the individual or respond to an inquiry from the individual and who can provide/explain the health profile related information to the individual. In certain embodiments, this person or provider can provide recommendations to the individual. For example, in selected embodiments, based at least in part on the health profile, the provider can tell the individual to see a doctor, can recommend lifestyle changes, can recommend support groups, can provide/suggest other care support services (e.g., as described in U.S. patent application Ser. No. 11/593,945, entitled HEALTHCARE MANAGEMENT SYSTEMS AND ASSOCIATED METHODS, filed Nov. 6, 2006), referenced above), and/or the like.

In still other embodiments, the health reporting component 212 can provide the related information to a healthcare provider associated with the individual (e.g., with permission, provide information to the individual's doctor). In still other embodiments, the reporting component 212 can summarize and report statistical information about a group of individuals (e.g., some or all of the portion of individuals that respond to health questions), for example, based on the related information of individuals in the group (e.g., 20% of the individuals in the group smoke). In yet other embodiments, the reporting component 212 can provide related information about one or more individuals to other systems or processes which can use the information to aid the individual(s), develop statistical data, etc. For example, in one embodiment a portion of the reporting component 212 can provide related information (e.g., as patient data) to a healthcare management process as described in U.S. patent application Ser. No. 11/593,945, entitled HEALTHCARE MANAGEMENT SYSTEMS AND ASSOCIATED METHODS, filed Nov. 6, 2006 (referenced above), which can provide care support services concurrently with, instead of, and/or subsequent to the reporting component 212 reporting information to the individual(s).

In still other embodiments, the health reporting component 212 can provide other information to one or more individuals along with information related to the one or more individuals' health profile. For example, in selected embodiments, the health reporting component 212 can provide motivational information and/or tools. For instance, in selected embodiments the health reporting component 212 can provide information to individual(s) about various incentive programs. For example, a cash reward for losing weight, a cash reward for going a selected number of days without smoking, time off for exercising a selected number of days per month, etc.

In selected embodiments, the readiness question generation component 214 can be configured to determine one or more individuals in the portion of individuals to provide a set of readiness questions (process portion 314), for example, based at least in part on a portion of the health profile of the one or more individuals. In other embodiments the readiness question generation component 214 can use other information, including the contact information, information gathered with the contact information, statistical data, and/or the like to formulate readiness questions/readiness question sets. The set of readiness questions can be formatted to assess a readiness of the one or more individuals to take a selected action. In certain embodiments, the readiness question generation component 214 can also be configured to contact and/or provide the set of readiness questions to the one or more individuals (process portion 314). In some embodiments, the readiness question generation component 214 can also be configured to provide information about the wellness program to individual(s) in a manner similar to that discussed above with reference to the health question generation component 204. In selected embodiments, the readiness question generation component 214 can be configured to generate or formulate the set of readiness questions for the one or more individuals based at least in part on a portion of the health profile of the one or more individuals (process portion 314).

For example, in certain embodiments the readiness questions can be formulated to determine the readiness or willingness of an individual to take an action related to his/her health profile (e.g., action to reduce a health risk, action to have a health condition monitored, action to seek/obtain treatment for a health condition, action to aid the individual in making a lifestyle change, action to aid an individual in dealing with a chronic or long term condition, etc.) For instance, in one embodiment where an individual's health profile indicates that the individual smokes, the readiness question generation component 214 can generate readiness questions to determine how ready and/or willing an individual is to take selected action(s) (e.g., to quit smoking). In certain embodiments, the responses or inputs to the readiness questions can be used to provide recommended course(s) of action, provide relevant information, provide suggestion(s), provide information about various resource(s), and/or the like. In certain embodiments, at least some individuals' health profiles may not indicate a need to provide readiness questions to those individuals.

In selected embodiments, the readiness question generation component 214 can include a single stored readiness question set that is provided to each individual for which information related to a health profile has been collected (e.g., questions stored in a database). In other embodiments, the readiness question generation component 214 can include multiple stored readiness question sets, and the readiness question generation component 214 can provide one or more selected readiness question sets to each individual based, at least in part, on the individual's associated health profile. In still other embodiments, the readiness question generation component 214 can include multiple stored readiness questions, and provide each individual with a selected readiness question set generated from the stored readiness questions based, at least in part, on the individual's associated health profile.

For example, in a selected embodiment where an individual has indicated that he or she smokes, the readiness question generation component 214 can ask the individual if he or she wants to quit smoking. If the individual answers yes, the readiness question generation component 214 can ask if the individual has tried to quit in the past. If the individual answers no, the readiness question generation component 214 can ask the individual if the individual believes that smoking is a major cause of lung cancer.

In selected embodiments, the readiness question generation component 214 can provide readiness questions (e.g., a set of readiness questions) to individuals via an automated telephone call or other computer media message that directs individual(s) to a website that provides the individual with the appropriate set of readiness questions once the individual provides selected identifying information (e.g., which can serve to verify the individuals identity). In still other embodiments, the readiness question generation component 214 can use an automated phone process or other computer media to provide a set of one or more readiness questions to an individual by providing a “call in” telephone number that presents readiness questions to the individual via an automated phone process. In other embodiments, the automated telephone call or other computer media can provide the individual with the capability of scheduling a time to receive an automated phone call during which the individual can respond to a readiness question set provided via the telephone system.

For example, in selected embodiments a computer media message can provide a hyperlink to a website that allows the individual to schedule a time to receive an automated phone call during which the individual can respond to readiness questions. In other embodiments, an automated telephone call can allow an individual to use touchtone keypad input(s) and/or voice recognition software to select a time to receive a second automated phone call (during which the individual can respond to readiness questions). In still other embodiments, the readiness question generation component 214 can simply contact the individual via an automated telephone process, allow the individual to verify their identity (e.g., via an employee ID or password), and provide the individual with readiness questions in a format that allows the individual to respond via the touchtone keypad on the phone and/or via voice recognition software.

In selected embodiments, the readiness input receiving component 216 can be configured to receive and/or store readiness input(s) (process portion 316), for example, from individuals in response to the set of readiness questions. For example, in certain embodiments one or more the individuals in the group of individuals can be provided readiness questions via an automated phone call (e.g., initiated by the computing system or via a “call in” number provided to the individual(s)) and the individual(s) can respond to at least a portion of the readiness questions (e.g., provide readiness inputs) via an automated telephone process (e.g., via touchtone keypad inputs and/or voice recognition software) which can be received and/or stored by the readiness input receiving component 216 (e.g., in a database).

In other embodiments, the readiness questions can be provided to one or more individuals via a computer generated display presented on a computer display device. The individual(s) can respond to the readiness questions (e.g., provide readiness inputs) using a computer input device. For example, the individual can respond to the readiness questions presented on a computer generated display (e.g., a webpage) or via speaker(s) using one or more computer input device such as a mouse, keyboard, keypad, and/or audio devices (e.g., using microphone(s) and voice recognition software). In selected embodiments, the readiness input receiving component 216 can receive and/or store these inputs.

In certain embodiments, a readiness input tracking component 218 can be configured to track readiness inputs (process portion 318) received from each individual. For example, in selected embodiments the readiness input tracking component 218 can be configured to determine one or more readiness questions in the set of readiness questions to which a selected individual has not responded. In selected cases, this can include determining that an individual has not answered any readiness questions, and/or has not scheduled a time to receive an automated call, has not accessed a “call in” phone number, has not visited a network site, and/or the like. In selected embodiments, the readiness input tracking component 218 can be further configured to prompt the selected individual to respond to, or provide input for, the one or more readiness questions to which the selected individual has not responded (process portion 318).

For example, the readiness input tracking component 218 can be configured to prompt a selected individual to answer any unanswered readiness questions before the selected individual exits/completes the set or series of readiness questions or readiness survey. For instance, the selected individual can select “submit” (e.g., via a phone input or via an input on a website) when the individual reaches the end of the survey. The readiness input tracking component 218 can prompt the individual to answer any unanswered readiness questions by highlighting the unanswered questions and/or presenting a new list of questions that includes the unanswered readiness questions (e.g., via the website or phone).

In other embodiments, once a selected individual has exited the survey without answering all of the readiness questions, the readiness input tracking component 218 can be configured to prompt the selected individual to return to the medium in which the readiness questions were originally presented (e.g., the same website and/or the same phone contact used to conduct the initial portion of the survey) to respond to any unanswered readiness questions. For example, in certain embodiments the readiness input tracking component 218 can send an email or place an automated call reminding the selected individual to return to the medium and complete the readiness survey and/or provide the individual with the option of completing the readiness survey at the time the reminder is received (e.g., via an automated phone process and/or a hyperlink to a website/network site). In selected embodiments, this feature can allow an individual to complete the readiness survey in parts (e.g., answering a portion of the readiness questions during a first time period and answering a second portion of the readiness questions during one or more subsequent time periods).

In still other embodiments, the readiness input tracking component 218 can prompt an individual to complete the readiness survey, but allow the individual to use a different medium than originally used. For example, in selected embodiments a selected individual can complete a portion of the readiness survey via a website during a first period of time. Subsequently (e.g., a few days later), the readiness input tracking component 218 can then initiate an automated phone call prompting the individual to complete the readiness survey via phone (e.g., by providing the unanswered readiness questions via phone and allowing the individual to respond via a touch tone keypad or voice recognition).

In selected embodiments, the readiness input tracking component 218 can prompt the individual to answer unanswered readiness questions, a portion of the readiness question generation component 214 can provide the questions, and a portion of the readiness input receiving component 216 can receive inputs provided in response to the unanswered readiness questions. In certain embodiments, the readiness input tracking component 218 can provide multiple prompts to an individual over a selected period of time, for example, when the individual does not answer all of the unanswered readiness questions after each prompt and/or does not respond to any of the prompts. In still other embodiments, after an individual fails to complete the readiness survey within a selected period of time and/or after a selected number of prompts, the readiness input tracking component 218 can provide notice to an operator or attendant who can follow up with the individual (e.g., via a person to person phone call).

In certain embodiments, a readiness analysis component 220 can be configured to analyzing readiness inputs (process portion 320) to determine the readiness of at least one individual to take at least one selected action. For example, in a selected embodiment where an individual smokes, the readiness analysis component 220 might determine from the readiness inputs that the individual does not want to quit and does understand the harmful effects of smoking. Accordingly, instead of providing the individual with information on smoking cessation classes, it might be better to provide the individual with information/education about the harmful effects of smoking and the effects it may have on the individual's family. In other cases, if the individual wants to quit smoking the appropriate treatment regimen might be to provide the individual with information on a smoking cessation programs and/or information about medication that the individual might get from a healthcare provider to help the individual to quit smoking.

A readiness reporting component 222 can be configured to report or provide information related to the readiness of at least one individual to take an action (process portion 322). For example, in selected embodiments information related to the readiness of an individual to take/perform a selected action can be reported to the individual via an automated phone call (e.g., after an appropriate identification and verification process) or via other computer media, such as an email, a password protected webpage, and/or the like. In other embodiments, the readiness reporting component 222 can provide information to an individual by providing a “call in” number or scheduling a call back time that will allow an individual to receive the readiness information via an automated phone process. In still other embodiments, the readiness reporting component 222 can automatically print mailers to the individuals to provide the associated/related information.

In yet other embodiments, the readiness reporting component 222 can report the related information to a person or provider who can call the individual or respond to an inquiry from the individual and who can provide/explain the readiness related information to the individual. In certain embodiments, this person or provider can provide recommendations to the individual. For example, in selected embodiments, based at least in part on the information related to the readiness of an individual to take/perform a selected action, the provider can tell the individual to see a doctor, can recommend lifestyle changes, can recommend support groups, can provide/suggest other care support services (e.g., as described in U.S. patent application Ser. No. 11/593,945, entitled HEALTHCARE MANAGEMENT SYSTEMS AND ASSOCIATED METHODS, filed Nov. 6, 2006), referenced above), and/or the like.

In still other embodiments, the readiness reporting component 222 can provide the related information to a healthcare provider associated with the individual (e.g., with permission, provide information to the individual's doctor). In still other embodiments, the readiness reporting component 222 can summarize and report statistical information about a group of individuals (e.g., some or all of the portion of individuals that respond to readiness questions), for example, based on the related information of individuals in the group (e.g., 75% of the individuals in the group that smoke are ready to quit smoking). In yet other embodiments, the readiness reporting component 222 can provide related information about one or more individuals to other systems or processes which can use the information to aid the individual(s), develop statistical data, etc. For example, in one embodiment a portion of the readiness reporting component 222 can provide related information (e.g., as patient data) to a healthcare management process as described in U.S. patent application Ser. No. 11/593,945, entitled HEALTHCARE MANAGEMENT SYSTEMS AND ASSOCIATED METHODS, filed Nov. 6, 2006 (referenced above), which can provide care support services concurrently with, instead of, and/or subsequent to the readiness reporting component 222 reporting information to the individual(s).

In yet other embodiments, the readiness reporting component 222 can provide other information to one or more individuals along with information related to the one or more individuals' readiness to take an action. For example, in selected embodiments, the readiness reporting component 222 can provide motivational information and/or tools. For instance, in selected embodiments the readiness reporting component 222 can provide information to individual(s) about various incentive programs. For example, a cash reward for losing weight, a cash reward for going a selected number of days without smoking, time off for exercising a selected number of days per week, etc.

Although the readiness question generation component 214, the readiness input receiving component 216, the readiness input tracking component 218, the readiness analysis component 220, and the readiness reporting component 222 have been discussed above in the context to readiness questions being based at least in part on a portion of the health profile of the one or more individuals that has been determined from a set of health questions, in other embodiments the readiness questions can be formulated based on other characteristics or considerations. For example, in other embodiments the readiness questions can be formulated to determine an individual's readiness to take an action based on general population norms and/or to take other types of actions. For example, in selected embodiments the readiness question generation component 214, the readiness input receiving component 216, the readiness input tracking component 218, the readiness analysis component 220, and/or the readiness reporting component 222 can be used to determine one or more individuals readiness to eat more fiber based on the assumption that most people do not eat enough fiber instead of the individual(s) actual health profile.

Some of the features described above can be used to efficiently and effectively administer at least a portion of a wellness program via one or more automated processes. Health and/or readiness related information gathered via some of the processes described above can be used to help individuals improve their health, reduce healthcare cost, reduce absenteeism, etc. Another feature of some of the embodiments described above is that the health and readiness question can be easily tailored for specific groups. For example, in certain embodiments the same health and readiness questions can be used for two different groups (e.g., groups of employees at two different companies), but a few questions can be modified, added, and/or deleted to address selected group characteristics. For instance, if one group has a high percentage of smokers, the health and readiness questions used for that group can be modified to focus more on smoking related health and readiness issues. In other embodiments, the health and readiness questions for different groups can be substantially (or totally) different.

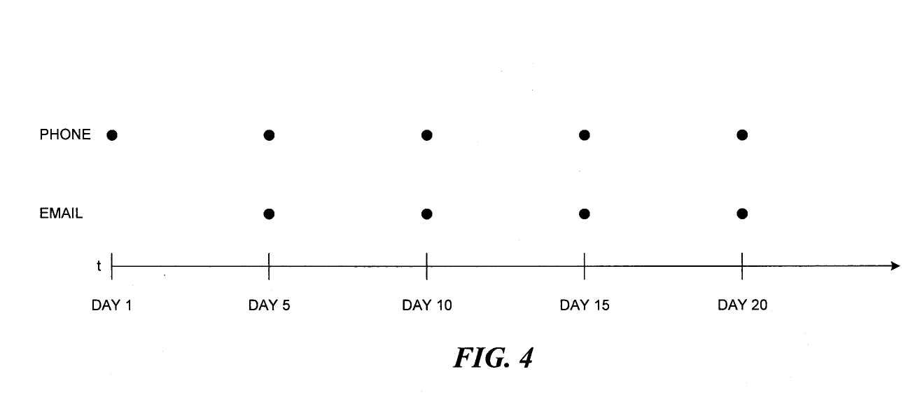

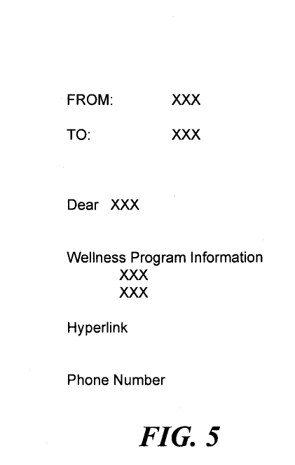

FIG. 4 is a partially schematic timeline of how some of the features discussed above can be used to administer at least a portion of a wellness program in accordance with an embodiment of the invention. In FIG. 4, on day 1 a computing system similar to the computing system discussed above with reference to FIG. 2 places an automated phone call to each individual of a company. The automated call provides general information about the wellness program and lets the individuals know what to expect. On day 5 the computing system places an automated phone call to each individual and provides the individuals with the option to answer health/readiness questions (via keypad entries and/or voice recognition software), to schedule a call back time to answer the questions, and/or to receive a network site (e.g., website) address which the individual can go to complete to answer health/readiness questions via the individuals personal computer. Additionally, the computing system automatically sends an email (similar to the one shown in FIG. 5) to each individual with information about the wellness program, a network address in the form of a hyperlink where the individual can answer health and readiness questions, and a phone number that the individual can call to answer health/readiness questions via an automated phone system.

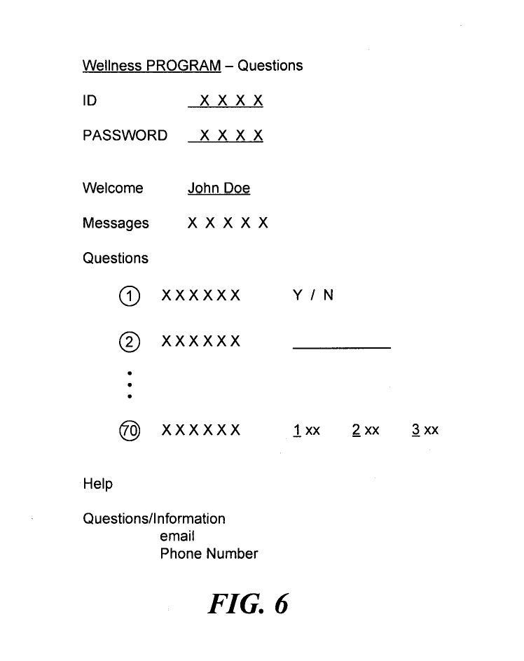

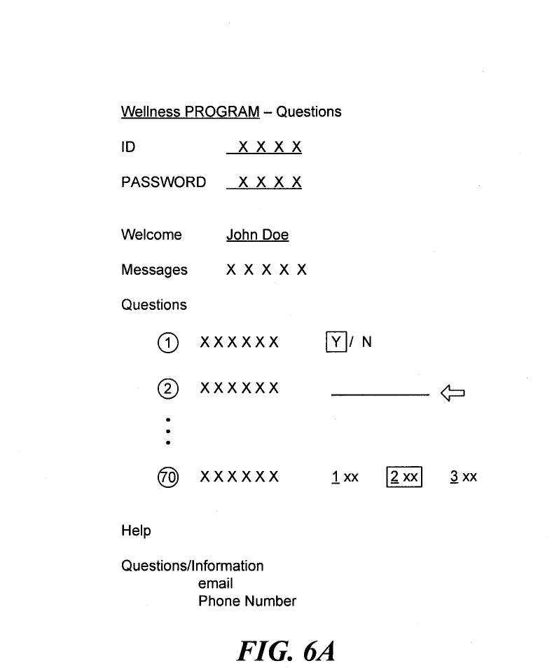

FIG. 6 is a partially schematic illustration of a display of a network site that provides questions in accordance with selected embodiments of the invention. For example, the individual can be directed to this site using the hyperlink in the email shown in FIG. 5. The network site can allow the individual to enter an ID and a password, provide a welcome message displaying the individual's name, and provide additional information about the wellness program. The network site can be interactive allowing the individual to answer the questions presented via the network site. For example, the network site can present a series of health questions and then a series of readiness questions (e.g., based at least in part on health profile information determined from the health questions), if appropriate. The display in FIG. 5 can also include a hyperlink that the individual can use to get help information (e.g., answers to frequently asked questions) and contact information (e.g., in the form of an email address and a phone number) which the individual can use to get additional information and/or ask questions.

On Day 10, the computing system can contact individuals that have not started and/or fully completed all of the appropriate health/readiness questions, reminding them that they have not completed all of the questions. For example, the computing system can contact the individual via the phone and via email in a manner similar to that discussed above with reference to day 5. As discussed above, the computing system can keep track of the questions which the individual has answered and not answered so that the individual only has to answer the unanswered questions. FIG. 6A is a partially schematic illustration of a display of a network site that provides an indication that not all of the questions shown in FIG. 6 have been answered in accordance with certain embodiments of the invention. In FIG. 6A, an arrow next to question 2 indicates that question 2 has not been answered (e.g., the individual identified as John Doe still needs to fill in the blank to answer question 2). As discussed above, in other embodiments other indicators can be used such as highlighting to indicate which questions remain unanswered. In still other embodiments, only unanswered questions are listed or shown on the display.

The computing system can contact the individuals that have not started and/or fully completed all of the appropriate health/readiness questions in a similar manner on days 15 and 20, as appropriate. For example, a selected individual that answers some questions on day 5, additional questions on day 10, and finishes all of the appropriate questions on day 15 would not receive a reminder on day 20. In some embodiments, individuals can use the call in number and or network site to complete the unanswered questions anytime after the first day (e.g., in between reminders).

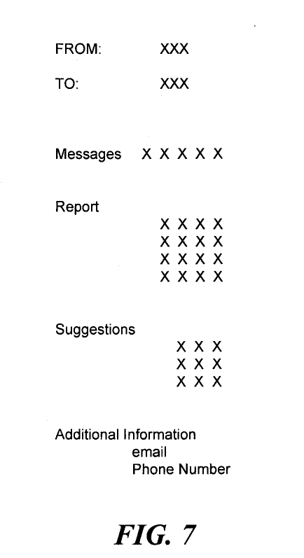

After an individual completes all health questions and/or all readiness questions, the computing system can analyze the associated inputs and provide a report to the individual (e.g., via an email, an automated phone call, a network site, an automatically produced mailer, etc.). For instance, FIG. 7 is a partially schematic illustration of an email automatically sent by the computing system to report health and readiness information to the individual. The email includes a general message containing information about the wellness program, reports information related to the individual's health profile and related to the individual's readiness to take one or more selected actions. Additionally, the email includes suggestions (e.g., recommendations, information, etc.) regarding the health and readiness related information. The email also includes an email and phone contact information where the individual can get additional information. Although in the illustrated embodiment the report includes information related to a health profile and information related to an individual's readiness to take one or more selected actions, in other embodiments the computing system can provide a report of information related to a health profile and/or a separate report of information related to an individual's readiness to take a selected action.

In selected embodiments, a wellness program can include providing a group of individuals (e.g., at a company) with multiple health and/or readiness surveys similar to those discussed above with reference to FIG. 4 (e.g., on an annual or semi-annual basis) in order to monitor changes in individual health and/or readiness information. Although the membership of the group can change slightly over time as individuals leave and/or join the group, in certain embodiments the health analysis component and/or the readiness analysis component can track the results associated with previous surveys for each individual. For example, these results can include the date of the surveys to which each individual has responded, how long each individual has been a member of the group, each individual's responses to the questions in the survey, and/or the like. Additionally, in selected embodiments the health analysis component and/or the readiness analysis component can use this information to monitor changes in individual health and/or readiness information (e.g., by comparing previous surveys to more recent surveys). The health reporting component and/or readiness reporting component can provide information related to changes in health or readiness information to the individual and/or other parties. In some cases, an incentive program can be used to reward positive changes. For example, in certain embodiments an individual can be given additional time off as a reward for taking certain steps to improve his or her health and/or for improving his or her health profile over time (e.g., as evidenced by the survey history).

The timeline in FIG. 4 and/or the displays in FIGS. 5-7 can have other configurations and/or other features. For example, in selected embodiments only an automated phone process is used to gather and present information. In other embodiments only email and network sites are used to gather and present information. Additionally, in other embodiments more, fewer, and/or different displays can be used by the computing system. In still other embodiments, the computing system includes more or fewer features. For example, in other embodiments the computing system only provides health questions to the individual(s).

From the foregoing, it will be appreciated that specific embodiments of the invention have been described herein for purposes of illustration, but that various modifications may be made without deviating from the invention. Additionally, aspects of the invention described in the context of particular embodiments may be combined or eliminated in other embodiments. For example, although advantages associated with certain embodiments of the invention have been described in the context of those embodiments, other embodiments may also exhibit such advantages. Furthermore, not all embodiments need necessarily exhibit such advantages to fall within the scope of the invention. Accordingly, the invention is not limited except as by the appended claims.

I/we claim:

1. A method in a computing system for administering at least a portion of a wellness program, the method comprising:
providing a set of health questions to each individual in a group of individuals, the set of health questions being formatted to determine a health profile of each individual that responds to the set of health questions;
receiving health inputs from the individuals in response to the set of health questions;
analyzing the health inputs to determine a health profile for each individual in a portion of individuals that respond to the set of health questions;
determining one or more individuals in the portion of individuals to provide a set of readiness questions based at least in part on a portion of the health profile of the one or more individuals, the set of readiness questions being formatted to assess a readiness of the one or more individual to take a selected action; and
providing the set of readiness questions to the one or more individuals.

2. The method of claim 1, further comprising:
receiving readiness input from at least one individual in response to the set of readiness questions;
analyzing the readiness input to determine the readiness of the at least one individual to take the selected action; and
provide information to the at least one individual related to the readiness of the at least one individual to take the selected action.

3. The method of claim 1, further comprising reporting information related to the health profile of the individuals in the portion of individuals that respond to at least a portion of the set of health questions.

4. The method of claim 1 wherein:
providing the set of health questions includes providing the set of health questions via at least one of a phone and a computer generated display; and
receiving the health inputs includes receiving health inputs via at least one of a phone and a computer input device.

5. The method of claim 1 wherein providing the set of readiness questions includes providing the set of readiness questions via at least one of a phone and a computer generated display, and wherein the method further comprises:
receiving readiness input from at least one individual in response to the set of readiness questions via at least one of a phone and a computer input device;
analyzing the readiness input to determine the readiness of the at least one individual to take the selected action; and
providing information to the at least one individual related to the readiness of the at least one individual to take the selected action.

6. The method of claim 1, further comprising:
tracking health inputs received from a selected individual;
determining one or more health questions in the set of health questions to which the selected individual has not responded; and
prompting the selected individual to provide input for the one or more health questions to which the selected individual has not responded.

7. The method of claim 1, further comprising formulating the set of readiness questions for the one or more individuals based at least in part on a portion of the health profile of the one or more individuals.

8. A computing system for administering at least a portion of a wellness program, comprising:
a health question generation component configured to provide a set of health questions to each individual in a group of individuals, the set of health questions being formatted to determine a health profile of each individual that responds to the set of health questions;
a health input receiving component configured to receive health inputs from the individuals in response to the set of health questions;
a health analysis component configured to analyze the health inputs to determine a health profile for each individual in a portion of individuals that respond to the set of health questions; and
a readiness question generation component configured to determine one or more individuals in the portion of individuals to provide a set of readiness questions based at least in part on a portion of the health profile of the one or more individuals, the set of readiness questions being formatted to assess a readiness of the one or more individuals to take a selected action, the readiness question generation component being configured to provide the set of readiness questions to the one or more individuals.

9. The system of claim 8, further comprising:
a readiness input receiving component configure to receive readiness input from at least one individual in response to the set of readiness questions;
a readiness analysis component configured to analyze the readiness input to determine the readiness of the at least one individual to take the selected action; and
a readiness reporting component configured to report information related to the readiness of the at least one individual to take the selected action.

10. The system of claim 8, further comprising:
a readiness input receiving component configure to receive readiness input from at least one individual in response to the set of readiness questions; and
a readiness input tracking component configured to track readiness inputs received from the one or more individuals and to determine one or more readiness questions in the set of readiness questions to which a selected individual not responded, the tracking component being further configured to prompt the selected individual to provide input for the one or more readiness questions to which the selected individual has not responded.

11. The system of claim 8, further comprising a health reporting component configured to report information related to the health profile of the individuals in the portion of individuals that respond to at least a portion of the set of health questions.

12. The system of claim 8 wherein:
providing the set of health questions includes providing the set of health questions via at least one of a phone and a computer generated display; and
receiving the health inputs includes receiving health inputs via at least one of a phone and a computer input device.

13. The system of claim 8 wherein providing the set of readiness questions includes providing the set of readiness questions via at least one of a phone and a computer generated display, and wherein the method further comprises:
a readiness input receiving component configured to receive readiness input from at least one individual in response to the set of readiness questions via at least one of a phone and a computer input device;
a readiness analysis component configured to analyze the readiness input to determine the readiness of the at least one individual to take the selected action; and
readiness reporting component configured to report information related to the readiness of the at least one individual to take the selected action.

14. The system of claim 8, further comprising a health input tracking component configured to track health inputs received from each individual and to determine one or more health questions in the set of health questions to which a selected individual has not responded, the tracking component being further configured to prompt the selected individual to provide input for the one or more health questions to which the selected individual has not responded.

15. The system of claim 8 wherein the readiness question generation component is configured to formulate the set of readiness questions for the one or more individuals based at least in part on a portion of the health profile of the one or more individuals.

16. A computing system for administering at least a portion of a wellness program, comprising:
a contact information receiving component configured to receive contact information for a group of individuals;
a health question generation component configured to contact each individual in the group of individuals and to provide a set of health questions to each individual, the set of health questions being formatted to determine a health profile of each individual that responds to the set of health questions;
a health input receiving component configured to receive health inputs from the individuals in response to the set of health questions;
a health input tracking component configured to track health inputs received from each individual and to determine one or more health questions in the set of health questions to which a selected individual has not responded, the tracking component being further configured to prompt the selected individual to provide input for the one or more health questions to which the selected individual has not responded;
a health analysis component configured to analyze the health inputs to determine a health profile for each individual in a portion of individuals that respond to the set of health questions; and
a health reporting component configured to report information related to the health profile of the individuals in the portion of individuals that respond to at least a portion of the set of health questions.

17. The system of claim 16 wherein the health question generation component is configured to contact each individual via at least one of a phone and a computer generated display.

18. The system of claim 16 further comprising:
a readiness question generation component configured to determine one or more individuals in the portion of individuals to provide a set of readiness questions based at least in part on a portion of the health profile of the one or more individuals, the set of readiness questions being formatted to assess a readiness of the one or more individual to take a selected action, the readiness question generation component being configured to provide the set of readiness questions to the one or more individuals;
a readiness input receiving component configure to receive readiness input from the one or more individuals in response to the set of readiness questions;
a readiness input tracking component configured to track readiness inputs received from the one or more individuals and to determine one or more readiness questions in the set of readiness questions to which a selected individual has not responded, the tracking component being further configured to prompt the selected individual to provide input for the one or more readiness questions to which the selected individual has not responded;
a readiness analysis component configured to analyze the readiness input to determine the readiness of at least one individual to take the selected action based on the readiness input received; and
a readiness reporting component configured to report information related to the readiness of the at least one individual to take the selected action.

19. The system of claim 16 further comprising a readiness question generation component configured to determine one or more individuals in the portion of individuals to provide a set of readiness questions based at least in part on a portion of the health profile of the one or more individuals, the set of readiness questions being formatted to assess a readiness of the one or more individual to take a selected action, the readiness question generation component being configured to provide the set of readiness questions to the one or more individuals.

20. The system of claim 16 wherein:
providing the set of health questions includes providing the set of health questions via at least one of a phone and a computer generated display; and
receiving the health inputs includes receiving health inputs via at least one of a phone and a computer input device.

21. A computing system for administering at least a portion of a wellness program, comprising:
a readiness question generation component configured to provide the set of readiness questions to the one or more, the set of readiness questions being formatted to assess a readiness of the one or more individual to take a selected action;
a readiness input receiving component configure to receive readiness input from the one or more individuals in response to the set of readiness questions;
a readiness analysis component configured to analyze the readiness input to determine the readiness of at least one individual to take the selected action based on the readiness input received; and
a readiness reporting component configured to report information related to the readiness of the at least one individual to take the selected action.

22. The system of claim 21, further comprising a readiness input tracking component configured to track readiness inputs received from the one or more individuals and to determine one or more readiness questions in the set of readiness questions to which a selected individual has not responded, the tracking component being further configured to prompt the selected individual to provide input for the one or more readiness questions to which the selected individual has not responded.

23. A computing system for administering at least a portion of a wellness program, comprising:
a health question generation component configured to provide a set of health questions to each individual in a group of individuals, the set of health questions being formatted to determine a health profile of each individual that responds to the set of health questions;
a health input receiving component configured to receive health inputs from the individuals in response to the set of health questions;
a health input tracking component configured to track health inputs received from each individual and to determine one or more health questions in the set of health questions to which a selected individual has not responded, the tracking component being further configured to prompt the selected individual to provide input for the one or more health questions to which the selected individual has not responded; and
a health analysis component configured to analyze the health inputs to determine a health profile for each individual in a portion of individuals that respond to the set of health questions.

24. The system of claim 23, further comprising a health reporting component configured to report information related to the health profile of the individuals in the portion of individuals that respond to at least a portion of the set of health questions.
